(19) United States
(12) Patent Application Publication
(10) Pub. No.: US 2024/0366546 A1
(43) Pub. Date: Nov. 7, 2024

(54) ANTI-HELICOBACTER PYLORI DERIVATIVE CONTAINING DIHYDROSTILBENE PARENT NUCLEUS AND PREPARATION METHOD AND APPLICATION THEREOF

(71) Applicant: NANJING UNIVERSITY OF CHINESE MEDICINE, Nanjing (CN)

(72) Inventors: Jinao DUAN, Nanjing (CN); Ming ZHAO, Nanjing (CN); Junfei ZHOU, Nanjing (CN); Hui YAN, Nanjing (CN); Sheng GUO, Nanjing (CN); Shulan SU, Nanjing (CN); Xinyi WEI, Nanjing (CN); Hongkai BI, Nanjing (CN)

(73) Assignee: NANJING UNIVERSITY OF CHINESE MEDICINE, Nanjing (CN)

(21) Appl. No.: 18/738,087

(22) Filed: Jun. 10, 2024

Related U.S. Application Data
(63) Continuation of application No. PCT/CN2024/080223, filed on Mar. 6, 2024.

(30) Foreign Application Priority Data
Jan. 10, 2024 (CN) 202410042684.5

(51) Int. Cl. A61K 31/216; A61K 31/05; A61K 31/085; A61K 31/11; A61K 36/484; A61P 31/04

(52) U.S. Cl. CPC A61K 31/216; A61K 31/05; A61K 31/085; A61K 31/11; A61K 36/484; A61P 31/04; A61K 2236/333; A61K 2236/39

(57) ABSTRACT

The invention belongs to the field of medicine, and particularly discloses an anti-Helicobacter pylori derivative containing a dihydrostilbene parent nucleus and a preparation method thereof; according to the invention, glycyrrhiza stems and leaves are used as raw materials, subjected to alcohol extraction, and then extracted, separated and identified through a macroporous resin, silica gel column chromatography and a preparative liquid phase to obtain a new natural isopentenyl dihydrostilbene compound, and the new compound has good activity to two kinds of drug-resistant and sensitive Helicobacter pylori, and can be used for preparing an anti-Helicobacter pylori drug.

### CROSS-REFERENCE TO RELATED APPLICATIONS

This application is the U.S. Continuing Application of International Application No. PCT/CN2024/080223 filed on 6 Mar. 2024 which designated the U.S. and claims priority to Chinese Application No. CN202410042684.5 filed on 10 Jan. 2024, the entire contents of each of which are hereby incorporated by reference.

### TECHNICAL FIELD

The present invention relates to a preparation method and anti-Helicobacter pylori activity of a dihydrostilbene compound, and belongs to the field of medicine.

### BACKGROUND

Helicobacter pylori is a gram-negative microaerophilic bacterium in a spiral or curved shape, which colonizes human gastric mucosa to cause chronic inflammation, and is an important pathogenic factor of chronic gastritis and gastroduodenal ulcer, and closely related to the occurrence of gastric cancer, gastric mucosa-associated lymphoid tissue lymphoma and other diseases. In 1994, the World Health Organization designated the Helicobacter pylori as a class I carcinogen of gastric cancer, and the Helicobacter pylori became the first officially recognized carcinogen in the world. An infection rate of the Helicobacter pylori in the global natural population is over 50%, and there is almost no chronic infectious disease in the world that will infect more than half of the global population. According to relevant statistical analysis, an infection rate of the Helicobacter pylori in China is as high as 55.82%, and a number of infected people are close to 800 Million. It is worth noting that China is a high incidence area of gastric cancer, and nearly a half of people suffering from the gastric cancer in the world are Chinese. Among the newly added cancer cases caused by pathogen infection in the world in 2018, there were 810,000 newly added cases of infection with the Helicobacter pylori in the world, with an incidence rate of 8.7/100,000, which exceeded those of human papillomavirus (8/100,000), hepatitis B virus (4.1/100,000) and hepatitis C virus (1.7/100,000), and in China, an incidence rate of cancer caused by the infection with the Helicobacter pylori was 15.6/100,000, which far exceeded the world average level.

In 2016, “the Fifth National Consensus Conference on the Management of Helicobacter Pylori Infection” pointed out that eradication treatment was recommended as long as Helicobacter pylori infection was confirmed by testing, regardless of symptoms and complications. Eradication of the Helicobacter pylori can not only promote the healing of peptic ulcer of body, reduce an incidence rate of ulcer complications, prevent or delay the atrophy of gastric mucosa and prevent the occurrence and development of intestinal metaplasia, but also reduce the incidence rate of gastric cancer by ⅓. At present, a bismuth quadruple therapy is recommended in China. However, due to the continuous emergence of drug-resistant strains of the Helicobacter pylori, it is increasingly difficult to eradicate the Helicobacter pylori with conventional treatment strategies, and it is urgent to find a new anti-Helicobacter pylori drug or lead compound.

Dried roots and rhizomes of glycyrrhiza (Glycyrrhiza uralensis Fisch., G. inflata Bat. and G. glabra L.) have the effects of invigorating spleen-stomach and replenishing qi, eliminating phlegm and relieving cough, detoxicating, relieving pain, and relieving drug properties, are used to treat spleen and stomach weakness, fatigue, palpitation and shortness of breath, cough with excessive phlegm, cramps and acute pains in abdomen and limbs, and carbuncle, swelling and sore-toxin, have a long history of relieving drug toxicity and potency, and are widely used in clinic and one of the commonly used medicinal materials. Glycyrrhiza stems and leaves are non-medicinal parts of glycyrrhiza, most of which are discarded in the fields, and are not reasonably used. Previous studies have shown that the glycyrrhiza stems and leaves are rich in isopentenyl compounds, and studies have shown that isopentenylation can enhance an antibacterial effect of compounds. To sum up, it is expected to obtain a novel efficient anti-Helicobacter pylori lead compound by carrying out ingredient separation.

### SUMMARY

The technical problem solved by the present invention is that: the present invention provides a preparation method of novel dihydrostilbene and an application of the dihydrostilbene in resisting Helicobacter pylori, and prepared compounds 1, 3, 6 to 7 and 9 all have good activity to two kinds of drug-resistant and sensitive Helicobacter pylori, and an MIC of 4 μg/mL to 8 μg/mL, and can be used for preparing an anti-Helicobacter pylori drug or used as lead compounds for developing the anti-Helicobacter pylori drug.

An anti-Helicobacter pylori derivative containing a dihydrostilbene parent nucleus comprises a compound with the following general structural formula:

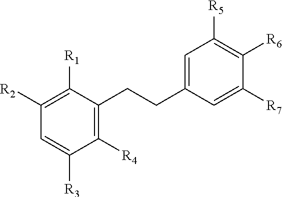

wherein R₁, R₂, R₃, R₄, R₅, R₆ and R₇ are all hydrogen, acyl, isopentenyl, hydroxyl, methoxyl, aldehyde or alkanoyloxy group.

Preferably, the anti-Helicobacter pylori derivative is selected from the group consisting of compound 1 having R₁=hydrogen, R₂=hydroxyl, R₃=isobutyryloxy, R₄=isopentenyl, R₅=hydrogen, R₆=hydroxyl, R₇=hydrogen; compound 2 having R₁=hydrogen, R₂=hydroxyl, R₃=2-methylbutyryloxy, R₄=isopentenyl, R₅=hydrogen, R₆=hydroxyl, R₇=hydrogen; compound 3 having R₁=hydrogen, R₂=methoxyl, R₃=hydroxyl, R₄=isopentenyl, R₅=hydrogen, R₆=hydroxyl, R₇=hydrogen; compound 4 having R₁=hydrogen, R₂=hydroxyl, R₃=acetyloxy, R₄=isopentenyl, R₅=isopentenyl, R₆=hydroxyl, R₇=hydrogen; compound 5 having R₁=hydrogen, R₂=hydroxyl, R₃=acetyloxy, R₄=isopentenyl, R₅=isopentenyl, R₆=methoxyl, R₇=hydroxyl; compound 6 having R₁=isopentenyl, R₂=hydroxyl, R₃=hydroxyl, R₄=isopentenyl, R₅=hydrogen, R₆=hydroxyl, R₇=hydrogen; compound 7 having R₁=isopentenyl, R₂=hydroxyl, R₃=acetyloxy, R₄=isopentenyl, R₅=hydrogen, R₆=hydroxyl, R₇=hydrogen; compound 8 having R₁=hydrogen, R₂=hydroxyl, R₃=hydroxyl, R₄=aldehyde group, R₅=hydrogen, R₆=hydroxyl, R₇=hydrogen; and compound 9 having R₁=hydrogen, R₂=hydroxyl, R₃=hydroxyl, R₄=isopentenyl, R₅=isopentenyl, R₆=hydroxyl, R₇=hydrogen.

A preparation method of the anti-Helicobacter pylori derivative containing the dihydrostilbene parent nucleus provided by the present invention comprises the following steps of:
(1) weighing and crushing medicinal materials of glycyrrhiza stems and leaves, adding ethanol or methanol solution to soak the medicinal materials at room temperature, heating, refluxing and extracting the mixture, filtering and then collecting and combining extracts, and concentrating the solution under a reduced pressure until no alcohol smell exists to obtain a concentrated solution of glycyrrhiza stems and leaves;
(2) loading the concentrated solution obtained in the step (1) on a macroporous resin column, and eluting the concentrated solution with ethanol solutions at different volume ratios to obtain six sub-fractions Fr.1-1 to Fr.1-5;
(3) loading the eluate sub-fraction Fr.1-4 obtained in the step (2) on a silica gel column, and eluting the eluate sub-fraction with petroleum ether and ethyl acetate mixed solvents at different volume ratios to obtain 13 sub-fractions Fr.2-1 to Fr.2-13;
(4) subjecting the eluate sub-fraction Fr.2-4 obtained in the step (3) to gradient elution by C₁₈-medium pressure preparation to obtain five sub-fractions Fr.3-1 to Fr.3-5; and subjecting the sub-fraction Fr.3-3 to gel column separation and purification by preparative high performance liquid chromatography to obtain a compound 3; and
(5) subjecting the eluate sub-fraction Fr.2-5 obtained in the step (3) to phase gradient elution by MCI-medium pressure preparation to obtain sixteen sub-fractions Fr.8-1 to Fr.8-16; subjecting the sub-fraction Fr.8-4 to preparative high performance liquid chromatography to obtain a compound 8; subjecting the sub-fraction Fr.8-8 to preparative high performance liquid chromatography to obtain a compound 6; subjecting the sub-fraction Fr.8-11 to preparative high performance liquid chromatography to obtain compounds 1 and 9; subjecting the sub-fraction Fr.8-12 to preparative high performance liquid chromatography to obtain compounds 2 and 7; and subjecting the sub-fraction Fr.8-13 to semi-preparative high performance liquid chromatography to obtain compounds 4 and 5.

Preferably, the preparation method of the anti-Helicobacter pylori derivative containing the dihydrostilbene parent nucleus above comprises the following steps of:
(1) weighing and crushing the medicinal materials of glycyrrhiza stems and leaves, adding the ethanol or methanol solution to soak the medicinal materials at room temperature, heating, refluxing and extracting the mixture at 105° C. for 3 times, each time for 2 hours, filtering and then collecting and combining the extracts, and concentrating the solution under a reduced pressure until no alcohol smell exists to obtain the concentrated solution of glycyrrhiza stems and leaves;
(2) carrying out macroporous resin column chromatography on the concentrated solution obtained in the step (1), a column volume being 20 L, and subjecting the concentrated solution to gradient elution with a methanol solution at a volume concentration of 0 to 100% to obtain the sub-fractions Fr.1-1 to Fr.1-5;
(3) carrying out silica gel column chromatography on the sub-fraction Fr.1-4 obtained in the step (2), a column volume being 10 L, and eluting the sub-fraction with petroleum ether/ethyl acetate systems at volume ratios of 6:1, 3:1, 1:1, 1:2 and 0:1 to obtain the sub-fractions Fr.2-1 to Fr.2-13;
(4) subjecting the eluate sub-fraction Fr.2-4 obtained in the step (3) to gradient elution by C₁₈-medium pressure preparation, wherein pure water A-methanol B is taken as a mobile phase to carry out gradient elution with 45% to 100% B for 300 minutes at a flow rate of 25 mL/min, so as to obtain the five sub-fractions Fr.3-1 to Fr.3-5; and subjecting the sub-fraction Fr.3-3 to gel column (AB-8, 1.5 cm×210 cm) chromatography, eluting the sub-fraction with methanol, and subjecting the sub-fraction eluted with methanol to preparative high performance liquid chromatography, wherein pure water A-methanol B is taken as a mobile phase to carry out isocratic elution at a volume ratio of 28:32, so as to obtain the compound 3 within a time period of 76 minutes to 80 minutes; and
(5) The eluate sub-fraction Fr.2-5 obtained in the step (3) was subjected to MCI-medium pressure preparation, wherein pure water A-methanol B was taken as a mobile phase to carry out gradient elution with 45% to 100% B for 300 minutes at a flow rate of 25 mL/min, so as to obtain 16 sub-fractions Fr.8-1 to Fr.8-16; the sub-fraction Fr.8-4 was subjected to preparative high performance liquid chromatography, wherein pure water A-methanol B was taken as a mobile phase to carry out isocratic elution at a volume ratio of 45:55 for 65 minutes, so as to obtain the compound 8 within a time period of 52 minutes to 56 minutes; the sub-fraction Fr.8-8 was subjected to preparative high performance liquid chromatography, wherein pure water A-methanol B was taken as a mobile phase to carry out isocratic elution at a volume ratio of 57:43 for 90 minutes, so as to obtain the compound 6 within a time period of 76 minutes to 80 minutes; the sub-fraction Fr.8-11 was subjected to preparative high performance liquid chromatography, wherein pure water A-methanol B was taken as a mobile phase to carry out isocratic elution at a volume ratio of 61:39 for 70 minutes, so as to obtain the compounds 1 and 9 within a time period of 48 minutes to 60 minutes and a time period of 60 minutes to 64 minutes respectively; the sub-fraction Fr.8-12 was subjected to preparative high performance liquid chromatography, wherein pure water A-methanol B was taken as a mobile phase to carry out isocratic elution at a volume ratio of 61:39 for 80 minutes, so as to obtain the compounds 2 and 7 within a time period of 58 minutes to 62 minutes and a time period of 66 minutes to 71 minutes respectively; and the sub-fraction Fr.8-13 was subjected to semi-preparative high performance liquid chromatography, wherein pure water A-methanol B was taken as a mobile phase to carry out isocratic elution at a volume ratio of 68:32 for 70 minutes, so as to obtain the compounds 4 and 5 within a time period of 54 minutes to 58 minutes and a time period of 60 minutes to 63 minutes respectively.

Anti-Helicobacter pylori activity of each compound is evaluated by a broth microdilution method in an activity experiment, and metronidazole, levofloxacin, clarithromycin and vancomycin used in clinic are selected as positive control drugs. Experimental results show that the compounds 1, 3, 6 to 7 and 9 all have good activity to two kinds of drug-resistant and sensitive Helicobacter pylori, and an MIC of 4 μg/mL to 8 μg/mL, are equivalent to a positive drug metronidazole, and can be used for preparing an anti-Helicobacter pylori drug or used as lead compounds for developing the anti-Helicobacter pylori drug.

The present invention discovers the novel dihydrostilbene from the glycyrrhiza stems and leaves for the first time, and provides an extraction and separation technology, structural identification method and application in resisting Helicobacter pylori of the new compound.

### Beneficial Effects

(1) The present invention aims to provide a novel derivative containing a dihydrostilbene parent nucleus with an anti-Helicobacter pylori effect and a preparation method thereof.
(2) The present invention discovers a good inhibitory effect of dihydrostilbene on the Helicobacter pylori for the first time. The compounds 1, 3, 6 to 7 and 9 all have good activity to two kinds of drug-resistant and sensitive Helicobacter pylori, and an MIC of 4 μg/mL to 8 μg/mL; and can be used for preparing an anti-Helicobacter pylori drug or used as lead compounds for developing the anti-Helicobacter pylori drug.

### BRIEF DESCRIPTION OF THE DRAWINGS

FIG. 1 is a ¹H NMR graph of a compound 1 (500 MHz, Methanol-d₄)

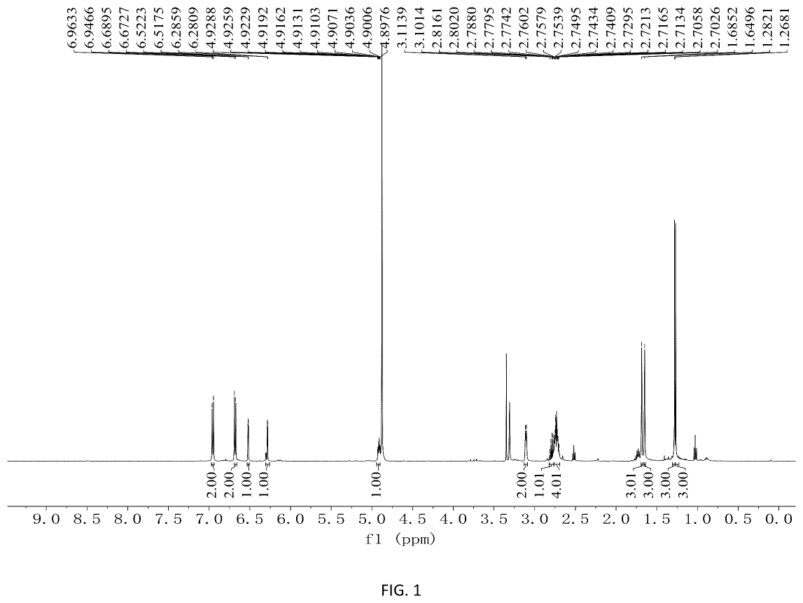

FIG. 2 is a ¹³C NMR graph of the compound 1 (125 MHz, Methanol-d₄)

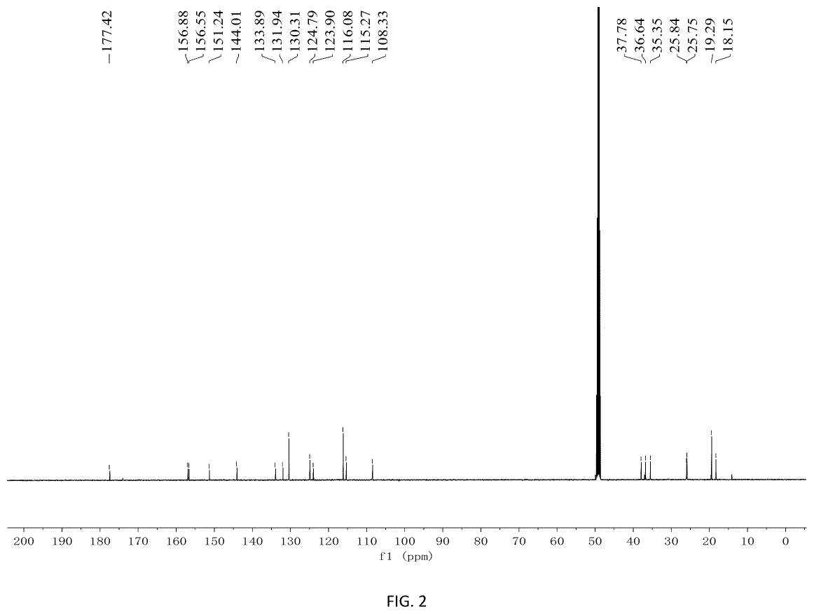

FIG. 3 is a ¹H NMR graph of a compound 2 (500 MHz, Methanol-d₄)

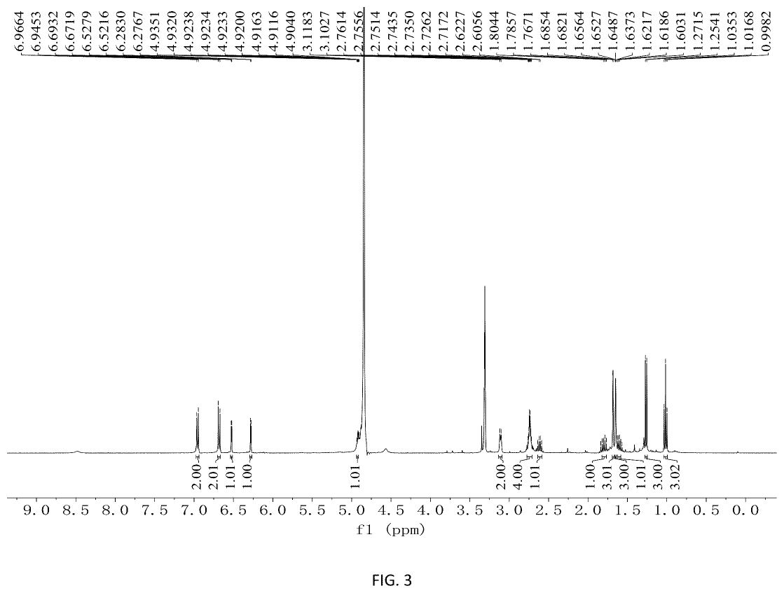

FIG. 4 is a ¹³C NMR graph of the compound 2 (125 MHz, Methanol-d₄)

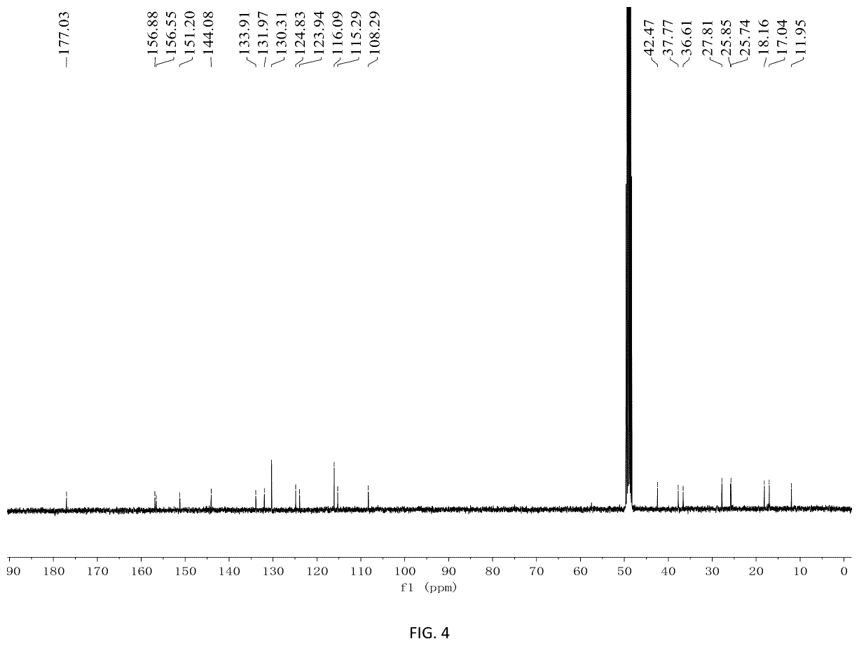

FIG. 5 is a ¹H NMR graph of a compound 3 (500 MHz, Methanol-d₄)

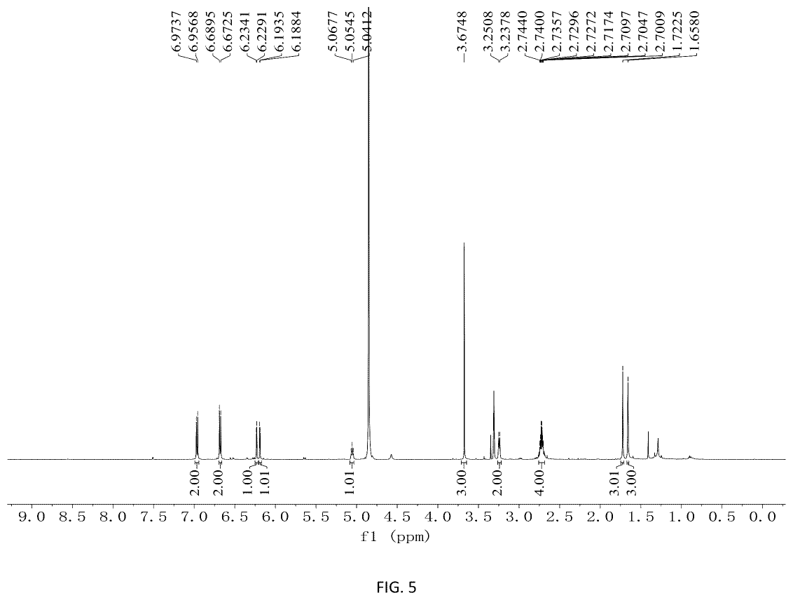

FIG. 6 is a ¹³C NMR graph of the compound 3 (125 MHz, Methanol-d₄)

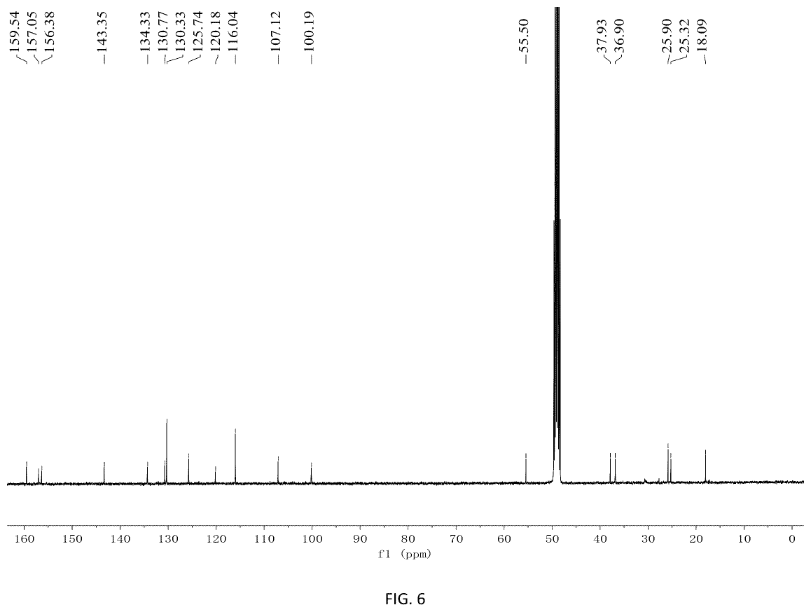

FIG. 7 is a ¹H NMR graph of a compound 4 (500 MHz, Methanol-d₄)

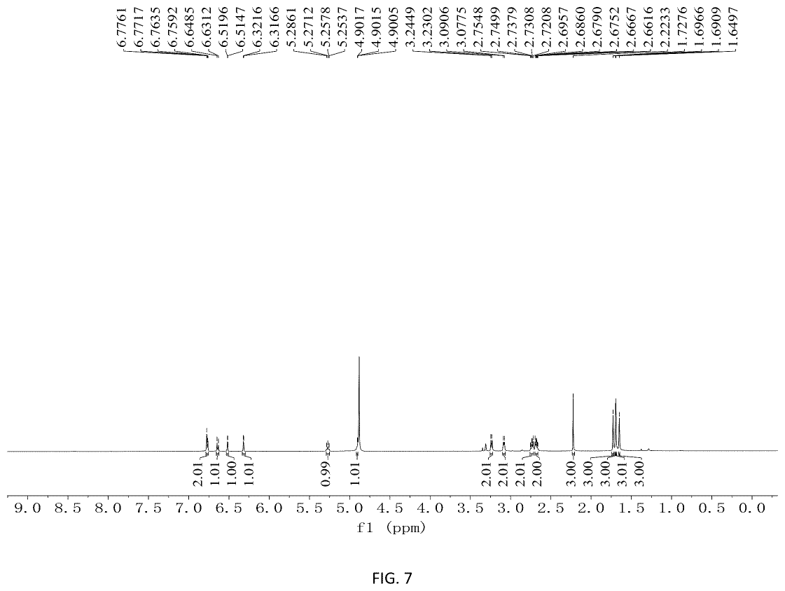

FIG. 8 is a ¹³C NMR graph of the compound 4 (125 MHz, Methanol-d₄)

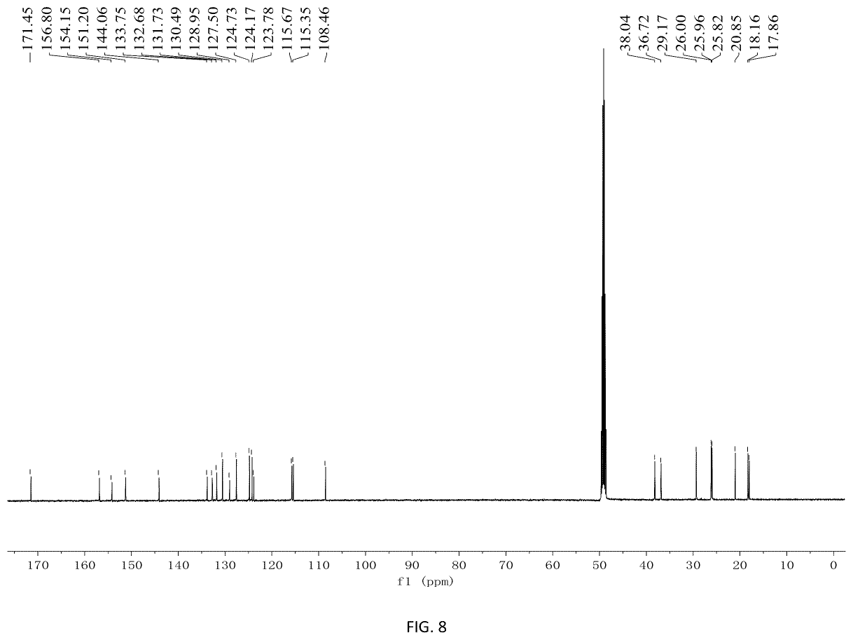

FIG. 9 is a ¹H NMR graph of a compound 5 (500 MHz, Methanol-d₄)

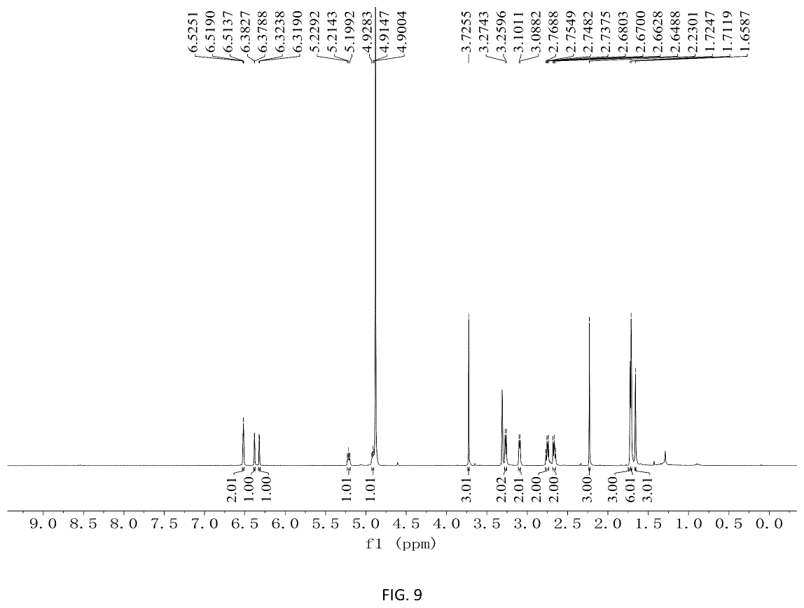

FIG. 10 is a ¹³C NMR graph of the compound 5 (125 MHz, Methanol-d₄)

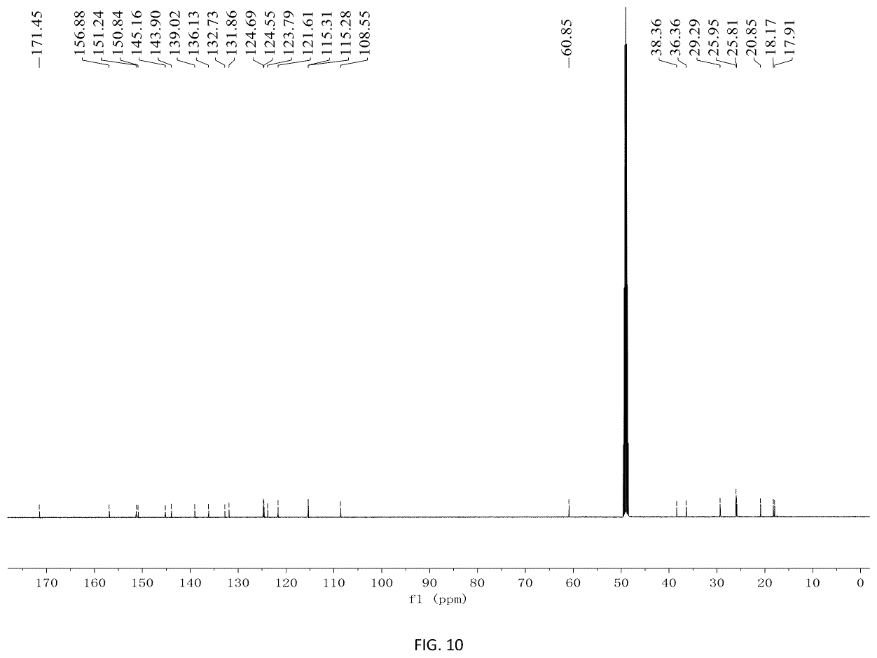

FIG. 11 is a ¹H NMR graph of a compound 6 (500 MHz, Methanol-d₄)

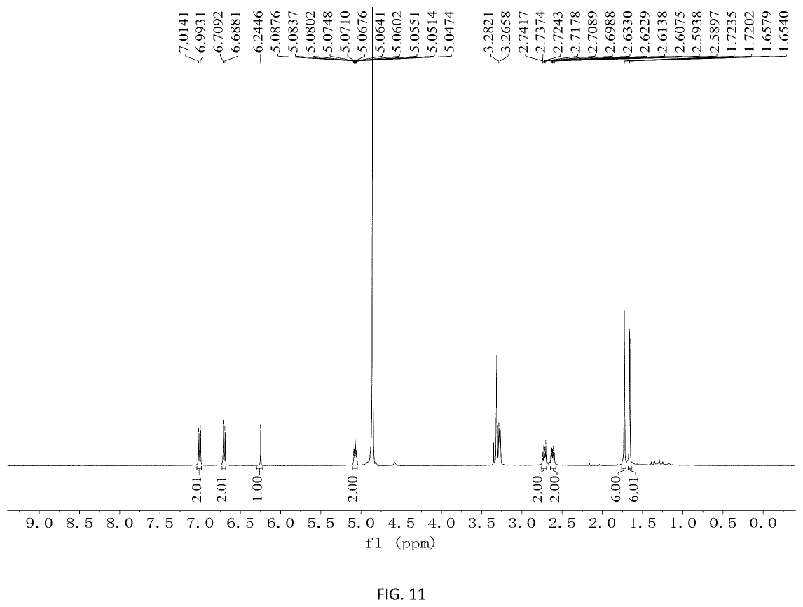

FIG. 12 is a ¹³C NMR graph of the compound 6 (125 MHz, Methanol-d₄)

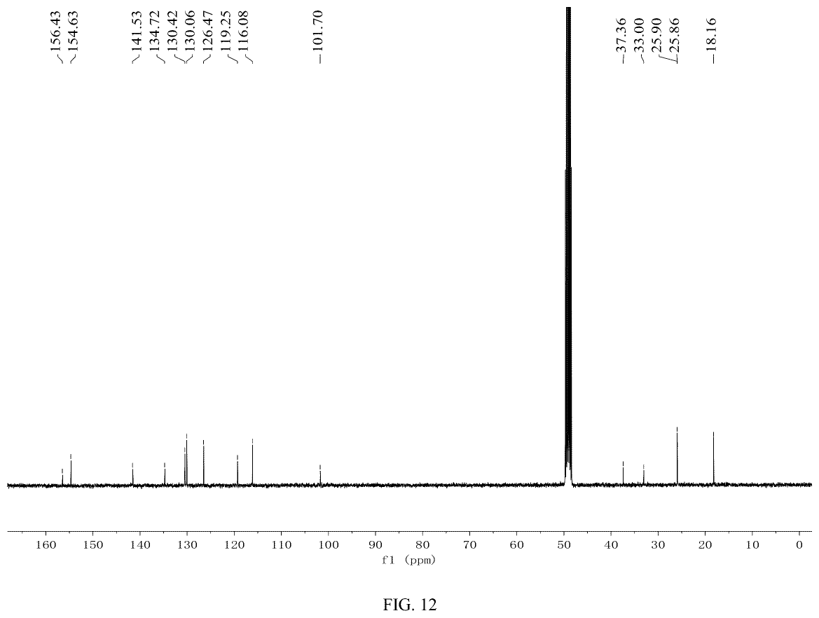

FIG. 13 is a ¹H NMR graph of a compound 7 (500 MHz, Methanol-d₄)

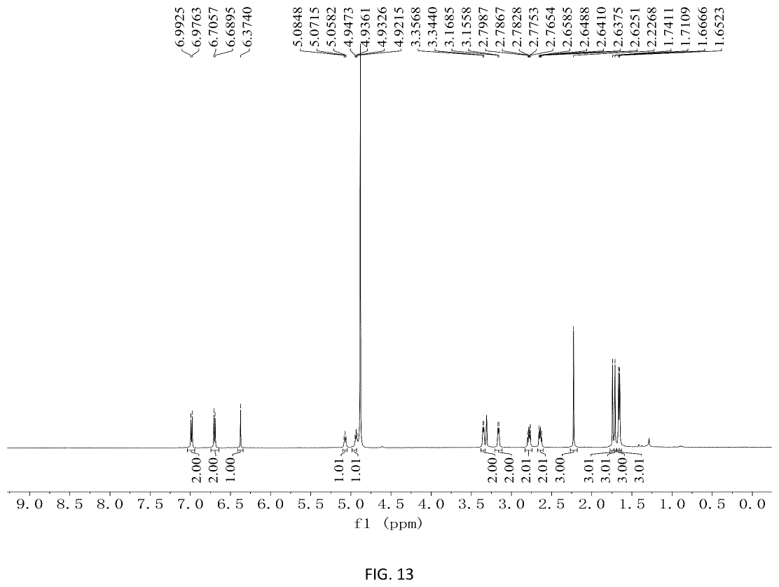

FIG. 14 is a ¹³C NMR graph of the compound 7 (125 MHz, Methanol-d₄)

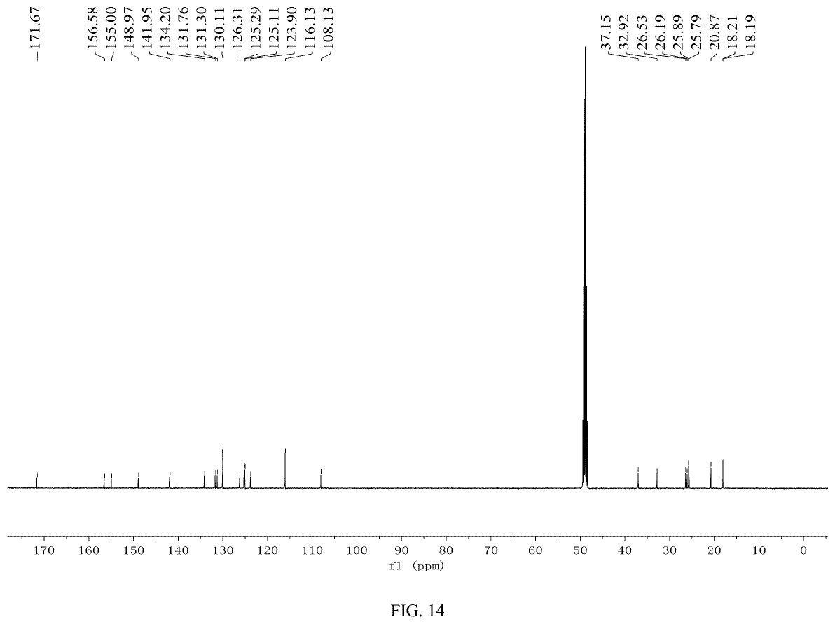

FIG. 15 is a ¹H NMR graph of a compound 8 (500 MHz, Methanol-d₄)

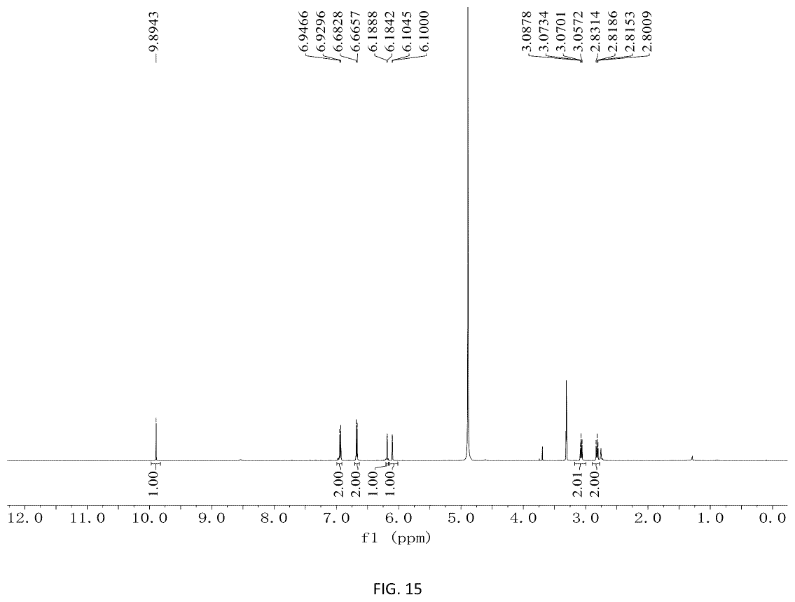

FIG. 16 is a ¹³C NMR graph of the compound 8 (125 MHz, Methanol-d₄)

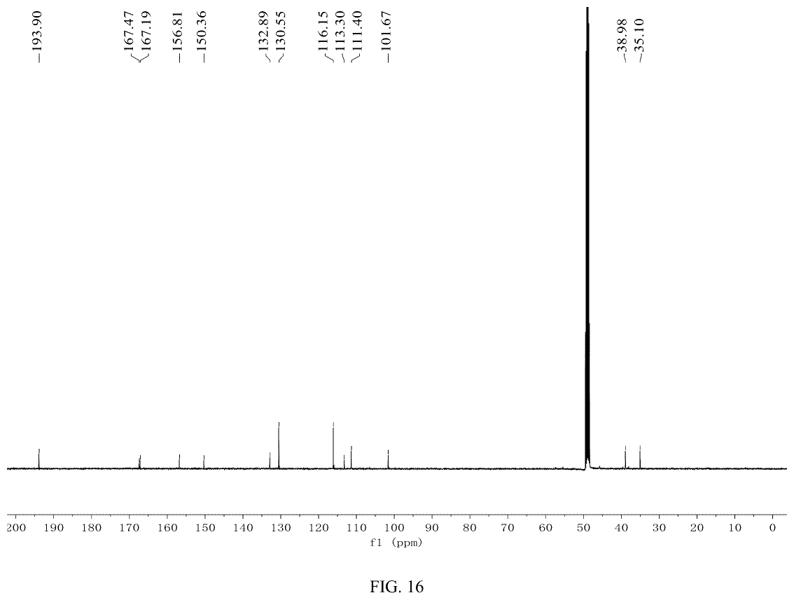

FIG. 17 is a ¹H NMR graph of a compound 9 (500 MHz, Methanol-d₄)

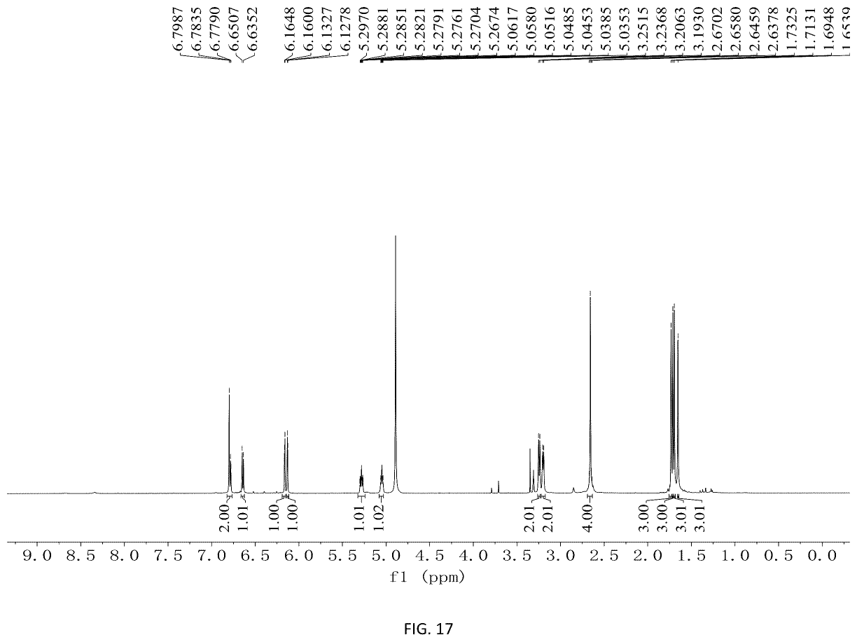

FIG. 18 is a ¹³C NMR graph of the compound 9 (125 MHz, Methanol-d₄)

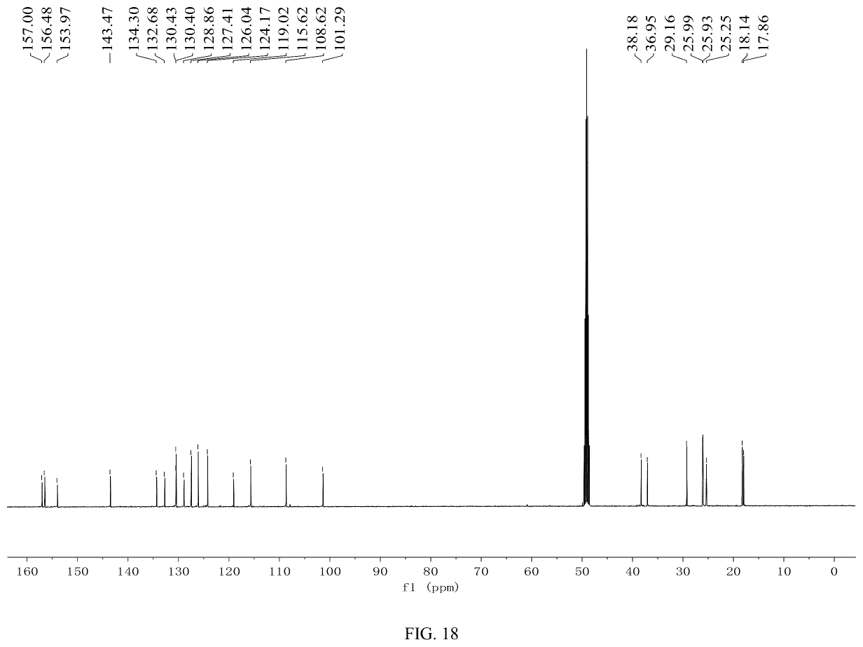

### DETAILED DESCRIPTION

The present invention can be better understood according to the following embodiments. However, it is easy for those skilled in the art to understand that specific material ratios, process conditions and results described in the embodiments are only used to illustrate the present invention, and shall not and will not limit the present invention described in detail in the claims.

### Embodiment 1

### 1. Instruments and Materials

### 1.1. Instruments

| Instrument | Manufacturer | Instrument | Manufacturer |
|---|---|---|---|
| ACQUITY UPLC high performance | Waters | S-B50L vertical | Jiangsu Jintan |
| liquid phase | Company of America | pressure steam | Medical Instrument |
| | | sterilizer | Factory |
| CHEETAH ®MP medium pressure | Tianjin Agela | Adjustable pipette | Eppendorf |
| preparative chromatography | Technologies Co., Ltd. | | Company of America |
| Nuclear magnetic resonance | Bruker | Microporous | Hangzhou Miu |
| instrument Bruker AV-500 | Company of Germany | plate thermostatic | Instruments Co., Ltd. |
| | | oscillator | |
| Shimadzu LC-20 AR preparative | Shimadzu | 7500 Real Time | Applied Biosystems |
| high performance liquid | Company of Japan | PCR System | Company of America |
| chromatographic instrument | | | |
| R-300 rotary evaporator | BUCHI | IS-RDV1 | Crystal |
| | Limited Company | constant temperature | Technology & Industries, |
| | of Switzerland | oscillator | Inc. of America |
| Intelligent artificial | Ningbo Saifu | PrimoStar inverted | ZEISS |
| climate box PRX-150B | Experimental | microscope | Company of Germany |
| | Instrument Co., Ltd. | | |
| Quitix 125 D-1CN electronic | Sartorius | SIM-F140AY65-PC | Panasonic Co., |
| scale | Scientific | ice machine | Ltd. of Japan |
| | Instruments Co., Ltd. | | |
| Milli-Q Advantage system | Millipore | Forma series II | Thermo |
| ultrapure water instrument | Company of America | water jacket CO₂ | Company of America |
| | | incubator | |
| 1300 series A2 clean bench | Thermo | TDL-80-2B | Shanghai |
| | Company of America | centrifugal machine | Anting Co., Ltd. |
| EnSpire multifunctional | PerkinElmer | | |
| microplate reader | Company of America | | |

### 1.2. Experimental Materials

Hedera ODS preparative chromatographic column (10 nm, 5 μm, 10 mm×250 mm); Waters ACQUITY UPLC BEH C₁₈ (2.1 mmx 100 mm, 1.7 μm) chromatographic column; MCI GEL (CHP20, 75-150 μm), Sephadex LH-20 gel, and column chromatography silica gel (200 meshes-300 meshes); chromatographic acetonitrile, methanol and formic acid purchased from Merck Company of America; and analytical petroleum ether and ethyl acetate purchased from Nanjing Wanqing Chemical Reagent Co., Ltd.

A preparation method of the present invention comprised the following steps.
(1) 5 kg of medicinal materials of glycyrrhiza stems and leaves were weighed and crushed, 10 L of 75% ethanol solution was added to soak the medicinal materials at room temperature for 1 hour, the mixture was heated, refluxed and extracted at 105° C. for 3 times, each time for 2 hours, extracts were filtered and then collected and combined, and the solution was concentrated under a reduced pressure until no alcohol smell existed to obtain a concentrated solution of glycyrrhiza stems and leaves.
(2) Macroporous resin column chromatography was carried out on the concentrated solution obtained in the step (1), a column volume was 20 L, and 400 L of concentrated solution was subjected to gradient elution with a methanol/water (0 to 100%) system to obtain sub-fractions Fr.1-1 to Fr.1-5.
(3) Silica gel column chromatography was carried out on the sub-fraction Fr.1-4 obtained in the step (2), a column volume was 10 L, and the sub-fraction was eluted with petroleum ether/ethyl acetate systems at volume ratios of 6:1, 3:1, 1:1, 1:2 and 0:1 to obtain sub-fractions Fr.2-1 to Fr.2-13.
(4) The eluate sub-fraction Fr.2-4 obtained in the step (3) was subjected to gradient elution by C₁₈-medium pressure preparation, wherein pure water A-methanol B was taken as a mobile phase to carry out gradient elution with 45% to 100% B for 300 minutes at a flow rate of 25 mL/min, so as to obtain five sub-fractions Fr.3-1 to Fr.3-5; and the sub-fraction Fr.3-3 was subjected to gel column (AB-8, 1.5 cm×210 cm) chromatography, the sub-fraction was eluted with methanol, and the sub-fraction eluted with methanol was subjected to preparative high performance liquid chromatography, wherein pure water A-methanol B was taken as a mobile phase to carry out isocratic elution at a volume ratio of 28:32, so as to obtain a compound 3 within a time period of 76 minutes to 80 minutes.
(5) The eluate sub-fraction Fr.2-5 obtained in the step (3) was subjected to MCI-medium pressure preparation, wherein pure water A-methanol B was taken as a mobile phase to carry out gradient elution with 45% to 100% B for 300 minutes at a flow rate of 25 mL/min, so as to obtain 16 sub-fractions Fr.8-1 to Fr.8-16; the sub-fraction Fr.8-4 was subjected to preparative high performance liquid chromatography, wherein pure water A-methanol B was taken as a mobile phase to carry out isocratic elution at a volume ratio of 45:55 for 65 minutes, so as to obtain a compound 8 within a time period of 52 minutes to 56 minutes; the sub-fraction Fr.8-8 was subjected to preparative high performance liquid chromatography, wherein pure water A-methanol B was taken as a mobile phase to carry out isocratic elution at a volume ratio of 57:43 for 90 minutes, so as to obtain a compound 6 within a time period of 76 minutes to 80 minutes; the sub-fraction Fr.8-11 was subjected to preparative high performance liquid chromatography, wherein pure water A-methanol B was taken as a mobile phase to carry out isocratic elution at a volume ratio of 61:39 for 70 minutes, so as to obtain compounds 1 and 9 within a time period of 48 minutes to 60 minutes and a time period of 60 minutes to 64 minutes respectively; the sub-fraction Fr.8-12 was subjected to preparative high performance liquid chromatography, wherein pure water A-methanol B was taken as a mobile phase to carry out isocratic elution at a volume ratio of 61:39 for 80 minutes, so as to obtain compounds 2 and 7 within a time period of 58 minutes to 62 minutes and a time period of 66 minutes to 71 minutes respectively; and the sub-fraction Fr.8-13 was subjected to semi-preparative high performance liquid chromatography, wherein pure water A-methanol B was taken as a mobile phase to carry out isocratic elution at a volume ratio of 68:32 for 70 minutes, so as to obtain compounds 4 and 5 within a time period of 54 minutes to 58 minutes and a time period of 60 minutes to 63 minutes respectively.

### Structural Analysis of Compounds

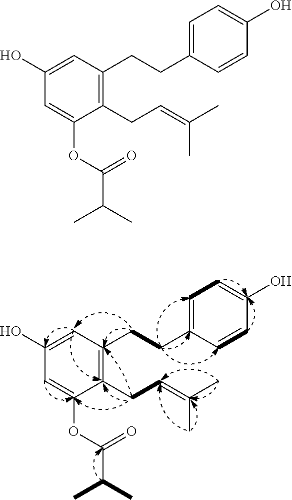

The compound 1 was an orange oil substance, and had an optical rotation value of [α]_{D}²⁰, +5.0 (c 0.08, MeOH); and HR-ESI-MS m/z 369.2066 [M+H]⁺, a calculated value of C₂₃H₂₉O₄⁺ was 369.2066), it was prompted that a molecular formula was C₂₃H₂₈O₄, and there were 10 unsaturation degrees. The compound 1 was highly similar to 2-(3-methyl-2-butenyl)-3,5,4′-trihydroxy-bibenzyl. A carbon spectrum and a hydrogen spectrum of the compound were analyzed, and a group of hydrogen signals δ_{H} 2.80 dd, 1.28 d and 1.27 d and a group of carbon signals δ_{C} 177.4, 35.4, 19.3 and 19.3 of isobutyryl were observed. In an HMBC spectrum, δ_{H} 3.11 (H-7) was respectively related to 6c 144.0 (C-1), 123.9 (C-2) and 151.2 (C-3), so that 2-isopentenyl substitution was determined. 6c 151.2 (C-3) shifted to a high field, so that 3-O-isobutyryl substitution was determined. In conclusion, the structure of the compound was determined to be 2-isopentenyl-3-isobutyryl-5,4′-dihydroxystilbene. The compound 1 was a new natural product, and named Uralenisol F.

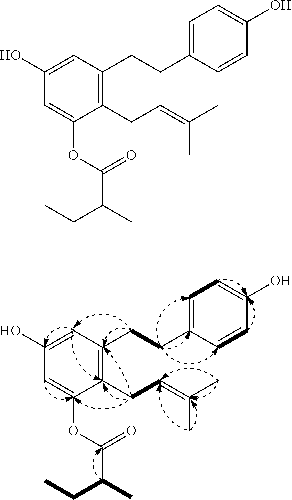

The compound 2 was an orange oil substance, and had an optical rotation value of [α]_{D}²⁰, +2.5 (c 0.08, MeOH); and HR-ESI-MS m/z 383.2215 [M+H]⁺, a calculated value of C₂₄H₃₁O₄+ was 383.2222), it was prompted that a molecular formula was C₂₄H₃₀O₄, and there were 10 unsaturation degrees. The compound 2 was highly similar to the compound 1. A carbon spectrum and a hydrogen spectrum of the compound were analyzed, and a group of feature signals δ_{H} 2.80 dd, 1.28 d and 1.27 d and a group of feature signals δ_{C} 177.4, 35.4, 19.3 and 19.3 of 2-methylbutyryl were observed. In an HMBC spectrum, δ_{H}3.11 (H-7) was respectively related to 6c 144.1 (C-1), 123.9 (C-2) and 151.2 (C-3), so that 2-isopentenyl substitution was determined. 6c 151.2 (C-3) shifted to a high field, so that 3-O-2-methylbutyryl substitution was determined. In conclusion, the structure of the compound was determined to be 2-isopentenyl-3-acetoxy-5,4′-dihydroxydihydrostilbene. The compound 2 was a new natural product, and named uralensisol G.

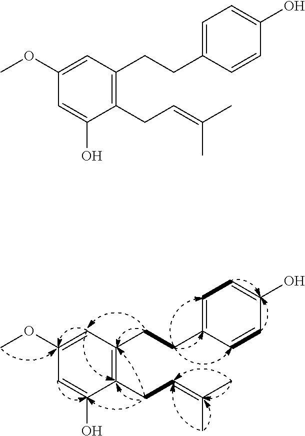

The compound 3 was an orange oil substance, and had an optical rotation value of [α]_{D}²⁰, +40.0 (c 0.08, MeOH); and HR-ESI-MS m/z 313.1818 [M+H]⁺, a calculated value of C₂₀H₂₅O₃⁺ was 313.1804), it was prompted that a molecular formula was C₂₀H₂₄O₃, and there were 9 unsaturation degrees. The compound 3 was highly similar to the compound 2-3-methyl-2-butenyl)-3,5,4′-trihydroxy-bibenzyl. There was a main difference that a hydrogen signal δ_{H} 3.67 (3H, s) of methoxyl existed in the compound 3. In an HSQC spectrum, δ_{H} 3.67 (3H, s) was directly related to δ_{C} 55.5, and in an HMBC spectrum, δ_{H}3.67 (3H, s) was remotely related to 6c 159.6, so that methoxyl substitution in a 5-position was determined. The compound 3 was a new natural product, and named uralensisol I.

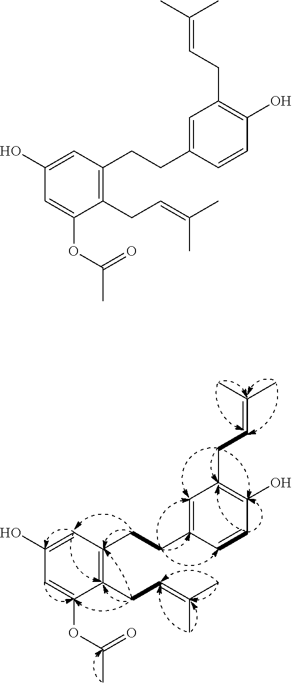

The compound 4 was an orange oil substance, and had an optical rotation value of [α]_{D}²⁰, +2.5 (c 0.08, MeOH); and HR-ESI-MS m/z 409.2385 [M+H]⁺, a calculated value of C₂₆H₃₃O₄⁺ was 409.2379), it was prompted that a molecular formula was C₂₆H₃₂O₄, and there were 11 unsaturation degrees. An A-ring of the compound 4 was similar to that of the compound 9. In addition, a ¹H NMR (500 MHz, Methanol-d₄) spectrum showed a group of hydrogen signals coupled with each other: δ_{H} 6.76 (dd, J=2.2, 8.7 Hz, H-6′), 6.64 (d, J=8.7 Hz, H-5′) and 6.77 (d, J=2.2 Hz, H-2′) and a group of hydrogen signals of isopentenyl: δ_{H} 3.24 (d, J=7.3 Hz, H-7′), 5.05 (t, J=7.3 Hz, H-5′), 1.65 (s, H-11′) and 1.70 (s, H-10′). In an HMBC spectrum, δ_{H} 3.24 (d, J=7.3 Hz, H-7′) was related to δ_{C} 154.2, 129.0 and 130.5, so that 3-isopentenyl-4′-hydroxy substitution of a B-ring was determined. δ_{C} 151.2 (C-3) shifted to a high field, and was related to δ_{C} 26.0 (C-7′), so that 3-O-acetyl substitution was determined. The compound 4 was a new natural product, and named uralensisol J.

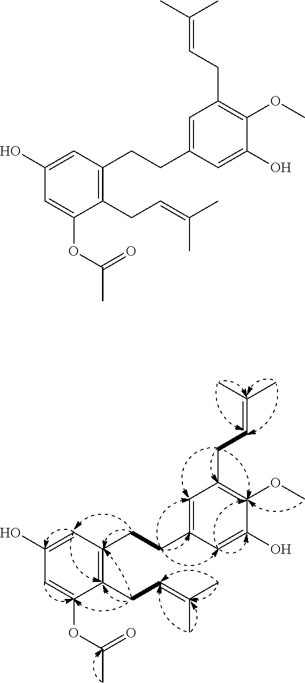

The compound 5 was an orange oil substance, and had an optical rotation value of [α]_{D}²⁰, −2.5 (c 0.08, MeOH); and HR-ESI-MS m/z 439.2488 [M+H]⁺, a calculated value of C₂₇H₃₅O₅⁺ was 439.2484), it was prompted that a molecular formula was C₂₇H₃₄O₅, and there were 11 unsaturation degrees. The compound 5 was highly similar to the compound 4. δ_{C} 145.2 (C-4′) and 150.8 (C-5′) signals in a ¹³C NMR (125 MHz, Methanol-d₄) spectrum were prompted to be O-diphenol hydroxyl substitution. In addition, a ¹H NMR (500 MHz, Methanol-d₄) spectrum showed a hydrogen signal δ_{H} 3.73 (3H, s) of methoxyl. In an HMBC spectrum, δ_{H} 3.73 (3H, s) was remotely related to δ_{C} 145.2, so that 4′-methoxyl substitution was determined. The compound 5 was a new natural product, and named uralensisol K.

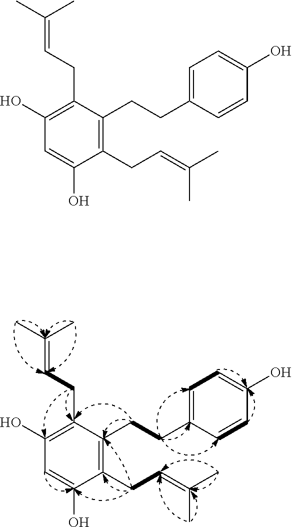

The compound 6 was an orange oil substance, and had an optical rotation value of [α]_{D}²⁰, +42.5 (c 0.08, MeOH); and HR-ESI-MS m/z 367.2278 [M+H]⁺, a calculated value of C₂₄H₃₁O₃⁺ was 367.2273), it was prompted that a molecular formula was C₂₄H₃₀O₃⁺, and there were 10 unsaturation degrees. The compound 6 was highly similar to gancaonin R. A main difference was that a B-ring of the compound 6 was subjected to non-o-diphenol hydroxyl substitution. In the compound 6, a ¹H NMR (500 MHz, Methanol-d₄) spectrum showed a group of AABB hydrogen signals δ_{H} 6.70 (2H, d, J=8.4 Hz) and δ_{H} 7.00 (2H, d, J=8.4 Hz). δ_{C} 130.1 (C-4′) and 116.1 (C-5′) signals in a ¹³C NMR (125 MHz, Methanol-d₄) spectrum were prompted to be 1,4 substitution. The compound 6 was a new natural product, and named uralensisol L.

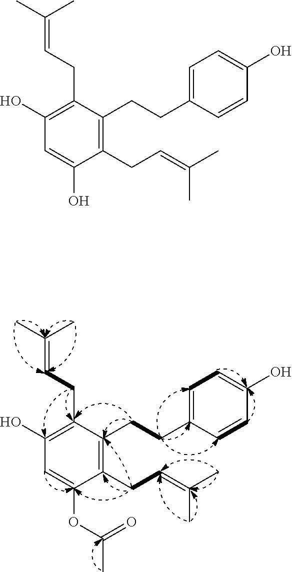

The compound 7 was an orange oil substance, and had an optical rotation value of [α]_{D}²⁰, +7.5 (c 0.08, MeOH); and HR-ESI-MS m/z 409.2379 [M+H]⁺, a calculated value of C₂₆H₃₃O₄⁺ was 409.2379), it was prompted that a molecular formula was C₂₆H₃₂O₄, and there were 10 unsaturation degrees. The compound 7 was highly similar to the compound 6. A main difference was that there was acetoxyl substitution in the compound 7. A ¹³C NMR (125 MHz, Methanol-d₄) spectrum showed feature signals δ_{C} 171.4 and 18.2 of acetyl. In addition, it was found that a 3-position carbon signal δ_{C} 149.0 (C-3) of the compound 7 shifted to a higher field than δ_{C} 154.6 (C-3) of the compound 6, so that acetoxyl substitution in a 3-position was determined. The compound 7 was a new natural product, and named uralensisol M.

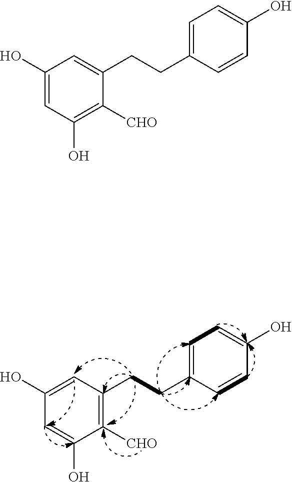

The compound 8 was an orange oil substance, and had an optical rotation value of [α]_{D}²⁰, +52.5 (c 0.08, MeOH); and HR-ESI-MS m/z 259.0970 [M+H]⁺, a calculated value of C₁₅H₁₅O₄⁺ was 259.0970), it was prompted that a molecular formula was C₁₅H₁₄O₄, and there were 9 unsaturation degrees. The compound 8 was highly similar to 3,5,4′-trihydroxy-bibenzyl. A main difference was that there was aldehyde group substitution in the compound 8. Hydrogen and carbon spectra of the compound both showed feature signals δ_{H} 9.89 (1H, s) and δ_{C} 193.9 of aldehyde group. In addition, it was found that a 3-position carbon signal δ_{C} 149.0 (C-3) of the compound 8 shifted to a higher field than δ_{C} 154.6 (C-3) of the compound 12, so that acetoxyl substitution in a 3-position was determined. The compound 8 was a new natural product, and named uralensisol N.

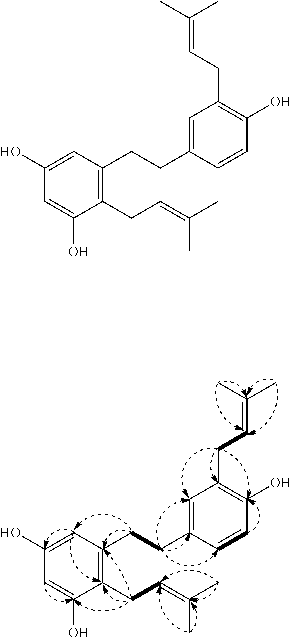

The compound 9 was an orange oil substance, and had an optical rotation value of [α]_{D}²⁰, +52.5 (c 0.08, MeOH); and HR-ESI-MS m/z 367.2275 [M+H]⁺, a calculated value of C₂₄H₃₁O₃⁺ was 367.2275), it was prompted that a molecular formula was C₂₄H₃₀O₃⁺, and there were 10 unsaturation degrees. The compound 9 was basically the same as the compound 4. There was no feature signal of acetyl in the compound 9, and δ_{C} 157.0 (C-3) of the compound shifted to a lower field than δ_{C} 151.2 (C-3) of the compound 10. Then, the structure of the compound was further determined by ¹H-¹H COSY, HSQC and HMBC. The compound 9 was a new natural product, and named uralensisol 0.

### Embodiment 2

An antibacterial activity test research of the present invention was carried out by the following steps.

### 1. Experimental Materials and Reagents

Helicobacter pylori strains HpG27, Hp159, Hp129 and Hp26695 provided by Professor Hongkai Bi of Nanjing Medical University. Culture mediums and main reagents: Colombian culture mediums, selective antibiotics (metronidazole, levofloxacin and clarithromycin), serum, and the like.

### 2. Preparation of Culture Medium

Colombian liquid culture medium: 29.0 g of Colombian liquid culture medium was accurately weighed, heated and dissolved in 1000 mL of double distilled water, and autoclaved at 121° C. for 15 minutes after the culture medium was completely dissolved for later use.

Colombian blood agar solid culture medium: 39.0 g of Colombian blood agar solid culture medium was accurately weighed, heated and dissolved in 1000 mL of double distilled water, autoclaved at 121° C. for 15 minutes after the culture medium was completely dissolved, naturally cooled to about 50° C. after the culture medium was autoclaved, quickly added with 5% sterile defibrinated sheep blood, mixed evenly, and poured into a sterile dish while the mixture was hot.

### Resuscitation and Culture of Tested Strains

The strains were taken out of a refrigerator at −80° C. and placed at room temperature, 200 L of standard strains were accurately sucked, transferred to a solid culture medium, and spread with an L-shaped glass rod, a culture dish loaded with the strains was put into a culture bag, then an AnaeroPack was put into the culture bag, and the culture bag was quickly sealed and then put into a constant temperature incubator at 37° C. to culture for 72 hours. After the end of culture, each strain was identified first, then a lawn on the solid culture medium was scraped off, and the lawn was transferred into 50 mL of liquid culture medium to serve as an original bacterial solution.

### Determination of MIC

(1) Sample solutions of the compounds 1 to 9 obtained by separation and purification above were prepared according to a concentration of 1 mg/mL.
(2) In preparation of an MIC plate, a first well was added with 176 μL of culture medium and then added with 3.2 μL of antibacterial drug, and dilution was carried out by multiple proportions to a 7^{th} well; and an 8^{th} well was not added with the drug, but was reserved with 90 μL of culture medium to serve as a control with bacteria but no drug.
(3) In preparation of a bacterial solution, Helicobacter pylori growing at a logarithmic phase on a solid plate was made into a bacterial suspension with a BHI culture medium, an OD₆₀₀ value of concentration of the bacterial suspension was adjusted to be 0.3 (1×10⁸ CFU/mL), and the bacterial suspension was diluted by 10 times to reach 1×10⁷ CFU/mL for later use.
(4) In inoculation, 10 μL of bacterial solution was added into the 1^{st} to 8^{th} wells (a concentration of the bacterial solution in each well was about 1.0×10⁶ CFU/mL). Results were judged after 72 hours of culture. Drug concentrations from the 1^{st} to 7^{th} wells were 16 μg/mL, 8 μg/mL, 4 μg/mL, 2 μg/mL, 1 μg/mL, 0.5 μg/mL and 0.25 μg/mL respectively.
(5) In result judgment, the lowest drug concentration capable of completely inhibiting the growth of bacteria in the wells was the MIC. The test was only meaningful when the bacteria in the 8^{th} well of the positive control well (without antibiotics) grew obviously. When there was a single drift in a microdilution method, the highest drug concentration capable of inhibiting the growth of bacteria should be recorded. If there were many drifts, the results should not be reported, and the test should be repeated. The test was repeated for 3 times for each drug.
(6) Specific experimental results were shown in Table 1.

TABLE 1
Minimum inhibitory concentrations of compounds
against helicobacter pylori (MIC: μg/mL)

| Compound | HpG27 | Hp26695 | Hp129 | Hp159 |
|---|---|---|---|---|
| 1 | 8 | 8 | 8 | 8 |
| 2 | >16 | >16 | >16 | >16 |
| 3 | 4 | 4 | 8 | 8 |
| 4 | >16 | >16 | >16 | >16 |
| 5 | >16 | >16 | >16 | >16 |
| 6 | 4 | 8 | 8 | 8 |
| 7 | 8 | 8 | 8 | 8 |
| 8 | >16 | >16 | >16 | >16 |
| 9 | 8 | 8 | 8 | 8 |
| MTZ | 2 | 1 | 32 (R) | 16 (R) |
| CLR | 0.008 | 0.008 | 1 (R) | 2 (R) |
| LVX | 0.25 | 0.25 | 8 (R) | 8 (R) |

MTZ, metronidazole; CLR, clarithromycin; and LVX, levofloxacin.

The present invention finds through activity screening that the compounds 1, 3, 6 to 7 and 9 all have good activity to two kinds of drug-resistant and sensitive Helicobacter pylori, and an MIC of 4 μg/mL to 8 ag/mL, are equivalent to a positive drug, and can be used for preparing an anti-Helicobacter pylori drug or used as lead compounds for developing the anti-Helicobacter pylori drug.

What is claimed is:

1. An anti-Helicobacter pylori derivative containing a dihydrostilbene parent nucleus, comprising a compound with the following general structural formula: 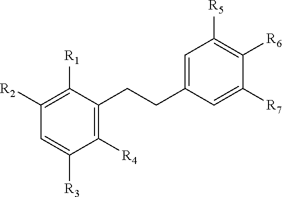
wherein R₁, R₂, R₃, R₄, R₅, R₆ and R₇ are selected from the group consisting of hydrogen, acyl, isopentenyl, hydroxyl, methoxyl, aldehyde and alkanoyloxy group, respectively.

2. The anti-Helicobacter pylori derivative containing the dihydrostilbene parent nucleus according to claim 1, where the anti-Helicobacter pylori derivative is selected from the group consisting of compound 1 having R₁=hydrogen, R₂=hydroxyl, R₃=isobutyryloxy, R₄=isopentenyl, R₅=hydrogen, R₆=hydroxyl, R₇=hydrogen; compound 2 having R₁=hydrogen, R₂=hydroxyl, R₃=2-methylbutyryloxy, R₄=isopentenyl, R₅=hydrogen, R₆=hydroxyl, R₇=hydrogen; compound 3 having R₁=hydrogen, R₂=methoxyl, R₃=hydroxyl, R₄=isopentenyl, R₅=hydrogen, R₆=hydroxyl, R₇=hydrogen; compound 4 having R₁=hydrogen, R₂=hydroxyl, R₃=acetyloxy, R₄=isopentenyl, R₅=isopentenyl, R₆=hydroxyl, R₇=hydrogen; compound 5 having R₁=hydrogen, R₂=hydroxyl, R₃=acetyloxy, R₄=isopentenyl, R₅=isopentenyl, R₆=methoxyl, R₇=hydroxyl; compound 6 having R₁=isopentenyl, R₂=hydroxyl, R₃=hydroxyl, R₄=isopentenyl, R₅=hydrogen, R₆=hydroxyl, R₇=hydrogen; compound 7 having R₁=isopentenyl, R₂=hydroxyl, R₃=acetyloxy, R₄=isopentenyl, R₅=hydrogen, R₆=hydroxyl, R₇=hydrogen; compound 8 having R₁=hydrogen, R₂=hydroxyl, R₃=hydroxyl, R₄=aldehyde group, R₅=hydrogen, R₆=hydroxyl, R₇=hydrogen; and compound 9 having R₁=hydrogen, R₂=hydroxyl, R₃=hydroxyl, R₄=isopentenyl, R₅=isopentenyl, R₆=hydroxyl, R₇=hydrogen.

3. The anti-Helicobacter pylori derivative containing the dihydrostilbene parent nucleus according to claim 1, wherein the anti-Helicobacter pylori derivative is prepared as a pharmaceutical drug with a pharmaceutically acceptable carrier.

4. The anti-Helicobacter pylori derivative containing the dihydrostilbene parent nucleus according to claim 3, wherein the pharmaceutical drug is prepared as a tablet, a pill, a capsule, an ointment, a powder, a mixture, an extract or a granule.

5. A method for preparing the anti-Helicobacter pylori derivative containing the dihydrostilbene parent nucleus according to claim 2, comprising the following steps of:
(1) weighing and crushing medicinal materials of glycyrrhiza stems and leaves, adding ethanol or methanol solution to soak the medicinal materials at room temperature, heating, refluxing and extracting the mixture, filtering and then collecting and combining extracts, and concentrating the solution under a reduced pressure until no alcohol smell exists to obtain a concentrated solution of glycyrrhiza stems and leaves;
(2) loading the concentrated solution obtained in the step (1) on a macroporous resin column, and eluting the concentrated solution with ethanol solutions at different volume ratios to obtain six sub-fractions Fr.1-1 to Fr.1-5;
(3) loading the eluate sub-fraction Fr.1-4 obtained in the step (2) on a silica gel column, and eluting the eluate sub-fraction with petroleum ether and ethyl acetate mixed solvents at different volume ratios to obtain 13 sub-fractions Fr.2-1 to Fr.2-13;
(4) subjecting the eluate sub-fraction Fr.2-4 obtained in the step (3) to gradient elution by C₁₈-medium pressure preparation to obtain five sub-fractions Fr.3-1 to Fr.3-5; and subjecting the sub-fraction Fr.3-3 to gel column separation and purification by preparative high performance liquid chromatography to obtain the compound 3; and
(5) subjecting the eluate sub-fraction Fr.2-5 obtained in the step (3) to phase gradient elution by MCI-medium pressure preparation to obtain sixteen sub-fractions Fr.8-1 to Fr.8-16; subjecting the sub-fraction Fr.8-4 to preparative high performance liquid chromatography to obtain a compound 8; subjecting the sub-fraction Fr.8-8 to preparative high performance liquid chromatography to obtain a compound 6; subjecting the sub-fraction Fr.8-11 to preparative high performance liquid chromatography to obtain the compounds 1 and 9; subjecting the sub-fraction Fr.8-12 to preparative high performance liquid chromatography to obtain the compounds 2 and 7; and subjecting the sub-fraction Fr.8-13 to semi-preparative high performance liquid chromatography to obtain the compounds 4 and 5.

6. The method according to claim 5, comprising the following steps of
(1) weighing and crushing the medicinal materials of glycyrrhiza stems and leaves, adding the ethanol or methanol solution to soak the medicinal materials at room temperature, heating, refluxing and extracting the mixture at 105° C. for 3 times, each time for 2 hours, filtering and then collecting and combining the extracts, and concentrating the solution under a reduced pressure until no alcohol smell exists to obtain the concentrated solution of glycyrrhiza stems and leaves;
(2) carrying out macroporous resin column chromatography on the concentrated solution obtained in the step (1), a column volume being 20 L, and subjecting the concentrated solution to gradient elution with a methanol solution at a volume concentration of 0 to 100% to obtain the sub-fractions Fr.1-1 to Fr.1-5;
(3) carrying out silica gel column chromatography on the sub-fraction Fr.1-4 obtained in the step (2), a column volume being 10 L, and eluting the sub-fraction with petroleum ether/ethyl acetate systems at volume ratios of 6:1, 3:1, 1:1, 1:2 and 0:1 to obtain the sub-fractions Fr.2-1 to Fr.2-13;
(4) subjecting the eluate sub-fraction Fr.2-4 obtained in the step (3) to gradient elution by C₁₈-medium pressure preparation, wherein pure water A-methanol B is taken as a mobile phase to carry out gradient elution with 45% to 100% B for 300 minutes at a flow rate of 25 mL/min, so as to obtain the five sub-fractions Fr.3-1 to Fr.3-5; and subjecting the sub-fraction Fr.3-3 to AB-8 gel column chromatography, eluting the sub-fraction with methanol, and subjecting the sub-fraction eluted with methanol to preparative high performance liquid chromatography, wherein pure water A-methanol B is taken as a mobile phase to carry out isocratic elution at a volume ratio of 28:32, so as to obtain the compound 3 within a time period of 76 minutes to 80 minutes; and
(5) subjecting the eluate sub-fraction Fr.2-5 obtained in the step (3) to MCI-medium pressure preparation, wherein pure water A-methanol B is taken as a mobile phase to carry out gradient elution with 45% to 100% B for 300 minutes at a flow rate of 25 mL/min, so as to obtain 16 sub-fractions Fr.8-1 to Fr.8-16; subjecting the sub-fraction Fr.8-4 to preparative high performance liquid chromatography, wherein pure water A-methanol B is taken as a mobile phase to carry out isocratic elution at a volume ratio of 45:55 for 65 minutes, so as to obtain the compound 8 within a time period of 52 minutes to 56 minutes; subjecting the sub-fraction Fr.8-8 to preparative high performance liquid chromatography, wherein pure water A-methanol B is taken as a mobile phase to carry out isocratic elution at a volume ratio of 57:43 for 90 minutes, so as to obtain the compound 6 within a time period of 76 minutes to 80 minutes; subjecting the sub-fraction Fr.8-11 to preparative high performance liquid chromatography, wherein pure water A-methanol B is taken as a mobile phase to carry out isocratic elution at a volume ratio of 61:39 for 70 minutes, so as to obtain the compounds 1 and 9 within a time period of 48 minutes to 60 minutes and a time period of 60 minutes to 64 minutes respectively; subjecting the sub-fraction Fr.8-12 to preparative high performance liquid chromatography, wherein pure water A-methanol B is taken as a mobile phase to carry out isocratic elution at a volume ratio of 61:39 for 80 minutes, so as to obtain the compounds 2 and 7 within a time period of 58 minutes to 62 minutes and a time period of 66 minutes to 71 minutes respectively; and subjecting the sub-fraction Fr.8-13 to semi-preparative high performance liquid chromatography, wherein pure water A-methanol B is taken as a mobile phase to carry out isocratic elution at a volume ratio of 68:32 for 70 minutes, so as to obtain the compounds 4 and 5 within a time period of 54 minutes to 58 minutes and a time period of 60 minutes to 63 minutes respectively.

7. A method for treating a disease comprising a step of administering a subject in need with an effective amount of the anti-Helicobacter pylori derivative of claim 1, wherein the disease is a Helicobacter pylori.

8. The method according claim 7, wherein the Helicobacter pylori is a drug-resistant or a sensitive Helicobacter pylori.
